(19) United States
(12) Patent Application Publication
(10) Pub. No.: US 2025/0290038 A1
(43) Pub. Date: Sep. 18, 2025

(54) METHOD FOR CONVERTING HUMAN SOMATIC CELLS INTO PROLIFERATIVE NEURAL STEM CELLS

(71) Applicant: KOREA UNIVERSITY RESEARCH AND BUSINESS FOUNDATION, Seoul (KR)

(72) Inventors: Sunghoi HONG, Seoul (KR); Yong NAM-KUNG, Seoul (KR)

(73) Assignee: KOREA UNIVERSITY RESEARCH AND BUSINESS FOUNDATION, Seoul (KR)

(21) Appl. No.: 18/858,543

(22) PCT Filed: Apr. 18, 2023
(86) PCT No.: PCT/KR2023/005222

§ 371 Date: Oct. 21, 2024

(30) Foreign Application Priority Data
Apr. 20, 2022 (KR) 10-2022-0049115

(51) Int. Cl. C12N 5/0797; A61K 35/30

(52) U.S. Cl. CPC C12N 5/0623; A61K 35/30; C12N 2501/11; C12N 2501/115; C12N 2501/999; C12N 2506/1307

(57) ABSTRACT

The present invention relates to a method of converting human fibroblasts into neural stem cells, and more particularly, to a conversion method through direct cross-differentiation of human fibroblasts into neural stem cells using a combination of Sendai virus, mRNA or miRNA of a stem cell-related factor, and a small molecule compound, and a use thereof. According to the present invention, since high-quality neural stem cells may be induced from human fibroblasts through direct cross-differentiation within a short period of time, it is possible to secure a sufficient amount of cells for cell therapy, and since there is no side effect of tumorigenesis, the present invention may be used as a cell therapeutic agent for brain diseases.

### TECHNICAL FIELD

The present invention relates to a method of converting human fibroblasts into neural stem cells, and more particularly, to a method of converting human fibroblasts into neural stem cells through direct cross differentiation using a combination of Sendai virus, mRNA or miRNA and a small molecule compound.

### BACKGROUND ART

As research on inducing human fibroblasts into induced pluripotent stem cells progressed in 2007, research on cell reprogramming began. Human embryonic stem cell-derived neural stem cells used in previous stem cell research have ethical issues arising from the use of human embryos, and issues with immune rejection, and the possibility of tumor formation when undifferentiated embryonic stem cells are transplanted. Adult stem cells have the problems that is it difficult to obtain cells and their differentiation ability is limited. However, although induced pluripotent stem cells are free from ethical issues and do not cause immune rejection, transplantation of undifferentiated stem cells can cause the problem of teratoma formation. In addition, direct cross-differentiated neural stem cells, which were published recently, have similar properties to induced pluripotent stem cells and embryonic stem-derived neural stem cells, but viral systems, which are a method mainly used to form neural stem cells, have the potential to form mutations through random insertion of genes. When transplanted into the human body, plasmids, proteins, and RNA are used to solve problems caused by viruses, but it may cause new, unidentified problems in terms of low differentiation efficiency into neural stem cells and the use of oncogenes.

As a solution to these problems with induced pluripotent stem cells, studies have been reported on the direct conversion of human fibroblasts into desired cells using a direct cross-differentiation method. Among them, direct differentiation of fibroblasts into neurons using fibroblasts for the treatment of intractable brain diseases has been actively studied, and research in which neurons were formed by introducing various combinations of neuron-related transcription factors into human fibroblasts was successful. This showed the possibility of being used as a cell therapeutic agent for intractable brain diseases, but it was difficult to obtain a sufficient amount of cells for cell therapy because they are already differentiated into neurons.

Due to these problems, recently, methods of directly differentiating neural stem cells using fibroblasts have been studied. For example, the methods include a method of inducing neural stem cells from fibroblasts by introducing various transcription factors using a viral system. The previous invention of the present inventors relating to a method of directly converting human fibroblasts into neural stem cells using a small molecule compound (PCT/KR2016/003819) suggested a method of inducing the process of directly converting fibroblasts into neural stem cells (direct cross-differentiation) using 8 types of small molecule compounds. In this direct cross-differentiation, there is a growing need for research on a method of proliferating sufficient amounts of neural stem cells to be used as a therapeutic agent by further shortening the differentiation period and increasing efficiency.

### DISCLOSURE

### Technical Problem

Therefore, while searching for a method of shortening the duration of direct cross-differentiation and generating high-efficiency, high-quality neural stem cells, the present inventors completed the present invention to enable proliferation in sufficient quantities for transplantation and induce genetically stable neural stem cells without tumorigenesis using one or more of Sendai virus, mRNA, and miRNA without insertion of a foreign gene; and a small molecule compound.

Accordingly, the present invention is directed to providing a direct cross-differentiation-inducing composition for inducing direct conversion into neural stem cells from fibroblasts, which includes one or more small molecule compounds selected from the group consisting of thiazovivin, valproic acid, purmorphamine, A8301, SB43154, CHIR99021, 5-aza-2′-deoxycytidine, and DZNep, and one or more of Sendai virus, mRNA, and miRNA.

The present invention is also directed to providing a method of preparing neural stem cells, which includes culturing somatic cells in a medium including one or more small molecule compounds selected from the group consisting of thiazovivin, valproic acid, purmorphamine, A8301, SB43154, CHIR99021, 5-aza-2′-deoxycytidine, and DZNep, and one or more of Sendai virus, mRNA, and miRNA.

The present invention is also directed to providing a cell therapeutic agent for treating a brain disease, including neural stem cells prepared by the above-described preparation method.

### Technical Solution

In one aspect, the present invention provides a direct cross-differentiation-inducing composition for inducing direct conversion from fibroblasts to neural stem cells, including one or more small molecule compounds selected from the group consisting of thiazovivin, valproic acid, purmorphamine, A8301, SB43154, CHIR99021, 5-aza-2′-deoxycytidine, and DZNep, and one or more of Sendai virus, mRNA, and miRNA.

In another aspect, the present invention provides a method of preparing neural stem cells, which includes culturing somatic cells in a medium including one or more small molecule compounds selected from the group consisting of thiazovivin, valproic acid, purmorphamine, A8301, SB43154, CHIR99021, 5-aza-2′-deoxycytidine, and DZNep, and one or more of Sendai virus, mRNA, and miRNA.

In still another aspect, the present invention provides a cell therapeutic agent for treating a brain disease, including neural stem cells prepared by the above-described preparation method.

Hereinafter, the present invention will be described in detail.

The present invention relates to a technology for preparing neural stem cells from fibroblasts using a combination of Sendai virus, mRNA or miRNA, and an optimal small molecule compound to induce neural stem cells from human fibroblasts.

To differentiate fibroblasts into neural stem cells without separate gene introduction, the present invention selected a direct cross-differentiation method, and to this end, the duration of differentiation may be shortened and efficiency may increase using a medium in which small molecule compounds for differentiation are combined and additionally using Sendai virus, mRNA, or miRNA to increase cross-differentiation efficiency. As in the present invention, through cross-differentiation using a small molecule material, and Sendai virus, mRNA or miRNA, various problems occurring when embryonic stem cells are used may be overcome, and the above-described composition may be used as a cell therapeutic agent with improved safety by reducing side effects (tumorigenesis, etc.) of a cell therapeutic agent using induced pluripotent stem cells.

“Neural stem cells” used herein refer to undifferentiated cells with pluripotency, which have self-replication ability and differentiate into neurons and/or glia, for example, astrocytes, oligodendrocytes and/or Schwann cells. Neural stem cells differentiate into neural cells, for example, neurons or glia, through the stage of neural progenitor cells or glial progenitor cells, which produce specific neural cells. The neural stem cells may then differentiate into one or more selected from the group consisting of an astrocyte, an oligodendrocyte, a neuron, a dopamine neuron, a GABA neuron, a motor neuron, and a choline neuron, but the present invention is not limited thereto.

“Direct cross-differentiation” is a cell reprogramming technology, and refers to a technology of directly reprogramming already mature differentiated cells to return them to stem cells. The present invention relates to a technology of reprogramming fibroblasts, which are human somatic cells, into neural stem cells through direct cross-differentiation, and since this is a process that directly differentiates into neural cells without going through the process of induced pluripotent stem cells, it can overcome the numerous disadvantages that occur during the differentiation of induced pluripotent stem cells and increase cell differentiation efficiency.

In one embodiment of the present invention, the present invention relates to a method of preparing neural stem cells for differentiation into neural stem cells through direct cross-differentiation by reprogramming human fibroblasts, which includes culturing human fibroblasts in a medium including one or more small molecule compounds selected from thiazovivin, valproic acid, purmorphamine, A8301, SB43154, CHIR99021, 5-aza-2′-deoxycytidine, and DZNep and one or more of Sendai virus, mRNA, and miRNA.

“Thiazovivin” used herein is known to block Rho/ROCK signaling inducing the apoptosis of neurons and neural stem cells and block PTEN signaling inhibiting the proliferation of neural stem cells, and is expected to inhibit the apoptosis and increase self-renewal and self-proliferation abilities of neural stem cells (Matthias Groszer, et al., Science 294:2186, 2001). Thiazovivin is a Rho-associated kinase (ROCK) inhibitor, which is a material serving to selectively inhibit ROCK. In addition to thiazovivin, Y-27632 may be used.

“VPA (valproic acid, 2-propylpentaonic acid)” used herein is a histone deacetylase inhibitor that inhibits histone deacetylase. It is known that VPA promotes the expression of cell proliferation inhibitors and genes necessary for differentiation induction by forming chromatin in a hyperacetylated state to induce cell (cancer cell) differentiation and inhibit angiogenesis, fixes the cell cycle in a Gl state to cause the apoptosis of cancer cells, thereby exhibiting high cytostatic anticancer activity. Histone deacetylase (HDAC) inhibits gene transcription via pRB/E2F, and the destruction of histone acetylation has been associated with the occurrence of various types of cancer. HDAC is highly expressed in harsh environmental conditions such as hypoxia, low sugar, and cell carcinogenesis and plays a role in promoting cell proliferation by inhibiting the expression of cell proliferation inhibitory factors, and is recognized as an important regulatory factor for cell carcinogenesis and differentiation regulation. As HDAC, trichostatin (TSA) or a derivative thereof may be used, in addition to VPA.

“Purmorphamine” used herein is a purine compound, and is known as a material that is involved in the Shh signaling system. The purmorphamine is not particularly limited as long as it can induce a Shh signal and may be one of various derivatives. For example, 2-(1-naphthoxy)-6-(4-morpholinoanilino)-9-cyclohexylpurin) may be purchased commercially and used. The purmorphamine may be treated in a medium commonly used to induce reprogramming to neural stem cell-like cells. Treatment with the Shh analog, purmorphamine, has the advantage that there is no need for gene introduction to produce neural stem cells from human fibroblasts.

“A-8301” used herein is a TGF-β type I receptor inhibitor and refers to a material that binds to a TGF-β type I receptor to inhibit the normal signaling process of TGF—B type I (Tojo M et al., CancerSci. 96:791-800, 2005). Transforming growth factor-β type I (TGF-β type I) is a multifunctional peptide involved in cell proliferation, differentiation and has various effects on various types of cells. Such multifunctionality is critical for the growth and differentiation of various types of tissue, such as adipogenesis, myogenesis, osteocytogenesis, and epithelial cell differentiation, and is known to inhibit the proliferation of neural stem cells. In addition to TGF-β type I receptor inhibitor A-8301, all TGF-β type I receptor inhibitors including SB432542 may be used, and the small molecule material, TGF-β I receptor inhibitor A-8301, may be purchased commercially or prepared, and neural stem cell proliferation is promoted by the treatment of the inhibitor.

“SB431542” used herein is an activin receptor-like kinase-5 (ALK5) inhibitor that induces fast reprogramming and improves chromosomal stability.

“CHIR99021” used herein is a glycogen synthase kinase (GSK) inhibitor that targets GSK1/2, which is an upstream molecule of GSK1/2 involved in the GSK signaling pathway. CHIR99021 is represented by aminopyrimidine, and in addition to CHIR99021, all GSK inhibitors may be used.

“5-Aza-2′-deoxycytidine” used herein also refers to decitabine, and is a cytidine analog that acts as a DNA demethylating agent. DNA demethylating agents participate in DNA replication and regulate gene expression by removing a methyl group (—CH₃) present on DNA. As a cytidine analog, such as decitabine, 5-azacytidine can be used.

“3-Deazaneplanocin A (DZNep)” used herein is an S-adenosylhomocysteine hydrolase inhibitor and histone methyltransferase EZH2 inhibitor, which induce apoptosis by turning on the suppressed tumor suppressor gene and inhibiting EZH2 expression, and prevent the EZH2 activation and trimethylation of lysine 27 on histone H3 in vitro. DZNep induces apoptosis in cancer cells, inhibits s-adenosylhomocysteine (SAH) hydrolase, reduces total DNA methylation, and improves Oct 4 expression in chemically induced pluripotent stem cells (CiPSCs).

In the present invention, for the differentiation of fibroblasts to neural stem cells, each small molecule is included at an effective concentration in a medium, and may be included by adjusting the effective concentration according to factors known in the art, such as the type of medium and the culture method.

The Sendai virus included in the medium for direct cross-differentiation of the present invention is for reprogramming of fibroblasts, which are somatic cells, and since it includes the Yamanaka factor, it can induce cells with multipotency. In one embodiment of the present invention, a CytoTune-IPS 2.0 Sendai reprogramming kit was used, and includes Sendai viruses containing Oct4, Sox2, Klf4, and C-myc. In the present invention, human fibroblasts were treated with the CytoTune-IPS 2.0 Sendai reprogramming kit, and reprogrammed for cross differentiation into neural stem cells.

In addition, in the present invention, the medium for differentiation of neural stem cells so was confirmed to have high cross differentiation efficiency compared to when a medium includes mRNA and a small molecule compound for OCT4 and/or SOX4 expression to induce direct cross-differentiation. OCT4 is a transcription factor required to maintain the pluripotency of stem cells, and SOX2 is known as a representative marker of neural stem cells and one of factors required when somatic cells are converted into induced pluripotent stem cells. In one embodiment of the present invention, an experiment to compare the results of inducing direct cross-differentiation into neural stem cells using an existing iNSC medium after regulating gene expression by transfecting human neonatal fibroblasts with OCT4 mRNA and SOX2 mRNA with that of the induction of direct cross-differentiation in the medium of the present invention was conducted.

In addition, direct cross-differentiation may be induced using miRNA302/367, and miRNA302/367 serves to inhibit the expression of the NR2F2 gene that inhibits OCT4, which is a gene for maintaining the pluripotency of stem cells. miRNA302/367 is an important cluster that suppresses NR2F2 to induce OCT4 expression and induce direct cross-differentiation into stem cells. Accordingly, in one embodiment of the present invention, differentiation efficiency was compared with that when direct cross-differentiation was induced in the medium of the present invention according to the experiment for inducing direct cross-differentiation into neural stem cells using the existing iNSC medium after regulating a gene by transfecting human Huntington skin fibroblasts with the miRNA302/307 cluster.

In addition, in the present invention, the medium includes all media conventionally used in culturing neural stem cells. The medium used in culture generally includes a carbon source, a nitrogen source, and trace elements. The medium may include, but is not limited to, DMEM/F12, N2, B27, a basic fibroblast growth factor (bFGF), and an epidermal growth factor (EGF).

The medium for the induced stem cell culture of the present invention may be any basal medium known in the art without limitation. The basal medium may be artificially synthesized, or may be a commercially available medium. Commercially available media include, for example, Dulbecco's modified Eagle's medium (DMEM), minimal essential medium (MEM), basal medium eagle (BME), RPMI 1640, F-10, F-12, α-minimal essential medium (α-MEM), Glasgow's minimal essential medium (G-MEM) and Iscove's modified Dulbecco's medium, but the present invention is not limited thereto.

In the present invention, the culture period of fibroblasts for direct cross-differentiation is preferably 10 to 20 days, and it was confirmed that fibroblasts differentiate into neural stem cells within 15 days. In one embodiment of the present invention, the neural stem cells were observed 13 days after culture, and it was confirmed that colonies of neural stem cells were formed between 15 to 19 days. In addition, immune staining showed that neural stem cells were differentiated through the expression of neural stem cell markers, such as Sox1, Sox2, MSH1, and Nestin.

In the present invention, the neural stem cells may maintain chromosomal stability, and maintain an undifferentiated state for 10 passages or more, but the present invention is not limited thereto.

In another embodiment of the present invention, the present invention relates to a cell therapeutic agent including neural stem cells cultured by the above-described method.

The cell therapeutic agent is a drug used for treatment, diagnosis and prevention using cells and tissue prepared by the isolation, culture, and recombination from humans, which means that cells are used to diagnose, prevent or treat a disease by genetic modification or a change in biological properties.

The cell therapeutic agent of the present invention may be a pharmaceutical composition for preventing or treating a brain disease.

The term “prevention” used herein refers to all actions that can inhibit vascular calcification or delay the onset thereof by administering the pharmaceutical composition according to the present invention.

The term “treatment” used herein refers to all actions involved in improving or beneficially changing symptoms by administering the pharmaceutical composition according to the present invention.

The pharmaceutical composition of the present invention may be used in the form of an oral formulation such as a powder, a granule, a tablet, a capsule, a suspension, an emulsion, a syrup, or an aerosol, a quasi-drug, a suppository, or a sterilized injectable solution according to a conventional method, and further include a carrier or excipient required for formulation. The pharmaceutically acceptable carrier, excipient and diluent, which are further added to the active ingredient, include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxybenzoate, magnesium stearate, and mineral oil. The pharmaceutical composition according to the present invention may be prepared using a diluent or an excipient such as a filler, a thickening agent, a binder, a wetting agent, a disintegrant, and a surfactant, which are commonly used.

For example, solid preparations for oral administration include tablets, pills, powders, granules, and capsules, and such solid preparations are prepared by mixing at least one excipient, such as starch, calcium carbonate, sucrose, lactose, or gelatin with the extract or compound. In addition, aside from simple excipients, lubricants such as magnesium stearate, talc, etc. are further used. Liquid preparations for oral administration include suspensions, liquids for internal use, emulsions, and syrups, and may further include various types of excipients, for example, a wetting agent, a sweetener, a fragrance and a preservative, other than a commonly-used simple diluent such as water, or liquid paraffin.

Preparations for parenteral administration include sterilized aqueous solutions, non-aqueous solvents, suspensions, emulsions, lyophilized formulations, and suppositories. As a non-aqueous solvent or suspension, propylene glycol, polyethylene glycol, a vegetable oil such as olive oil, or an injectable ester such as ethyl oleate may be used. As a base for the suppository, Witepsol, macrogol, Tween 61, cacao buffer, laurin butter, and glycerogelatin.

The pharmaceutical composition of the present invention may be administered orally or parenterally (e.g., intravenously, subcutaneously, percutaneously, nasally or intratracheally) according to a desired method, and a dose of the pharmaceutical composition of the present invention may be selected according to a patient's condition and body weight, severity of a disease, a dosage form, an administration route and duration by those of ordinary skill in the art. Particularly, since the pharmaceutical composition of the present invention includes a cell therapeutic agent, it can be administered by any device that allows the cell therapeutic agent to reach target cells.

The pharmaceutical composition may be administered at a pharmaceutically effective amount. The term “pharmaceutically effective amount” used herein refers to an amount sufficient for treating a disease at a reasonable benefit/risk ratio applicable for medical treatment, and an effective dosage may be determined by parameters including the type of a patient's disease, severity, drug activity, sensitivity to a drug, administration time, an administration route and an excretion rate, the duration of treatment, drugs simultaneously used, and other parameters well known in the medical field. The pharmaceutical composition of the present invention may be administered separately or in combination with other therapeutic agents, sequentially or simultaneously administered with a conventional therapeutic agent, or administered in a single or multiple dose(s). In consideration of all of the above-mentioned parameters, it is important to achieve the maximum effect with the minimum dose without side effects, and such a dose may be easily determined by one of ordinary skill in the art.

Specifically, the dose of the pharmaceutical composition may vary depending on a patient's age, body weight, severity, and sex, and may be administered at, for example, 1.0×10⁴ to 1.0×10¹⁰ cells/kg of body weight, and preferably, 1.0×10⁵ to 1.0×10⁹ cells/kg of body weight once or for in several divided doses. However, the actual dose of the active ingredient may be determined by various related factors, including a disease to be treated, the severity of a disease, an administration route, and the body weight, age, and sex of a patient, and the dose may be set differently as needed, but the present invention is not limited by the above-described contents.

### Advantageous Effects

The present invention relates to a method of converting human fibroblasts into neural stem cells, and more preferably, a method of converting human fibroblasts into neural stem cells through direct cross-differentiation using a combination of Sendai virus, mRNA or miRNA and a small molecule compound, and a use thereof. When human fibroblasts are directly converted into neural stem cells using the combination of Sendai virus, mRNA or miRNA, and a small molecule compound, the absence of gene insertion enables the induction of high-quality neural stem cells, and it is possible to obtain a sufficient amount of cells for cell therapy, and since there is no side effect of tumorigenesis, the present invention can be used as a cell therapeutic agent for brain diseases.

### DESCRIPTION OF DRAWINGS

FIG. 1 is a schematic diagram of the experiment for human neonatal fibroblasts to express Oct4. Sox2. Klf4, C-myc (or L-myc) gene without separate insertion using CytoTune-IPS 2.0 Sendai virus, and differentiate into neural stem cells using a small molecule compound.

FIG. 2 shows the results of observing the process of inducing the differentiation of human fibroblasts into neural stem cells by culturing them for 19 days with Sendai virus (SeV) and 8 types of small molecule compounds (8SMs).

FIG. 3 shows the experimental results for various combinations including small molecule compounds and Sendai virus to induce direct cross-differentiation of human neonatal fibroblasts into neural stem cells.

FIG. 4 shows the qRT-PCR results for stem cell markers of neural stem cells directly cross-differentiated from human neonatal fibroblasts.

FIG. 5 is a set of immunofluorescence staining images for identifying neural stem cell markers of neural stem cells directly cross-differentiated from human neonatal fibroblasts.

FIG. 6 shows the results of confirming chromosomal stability at the 2^{nd} and 10^{th} passages of subculture to confirm the genetic stability of induced neural stem cells.

FIG. 7 shows the culturing results (colonies) of neural stem cells induced by direct cross-differentiation from human adult fibroblasts.

FIG. 8 is a set of immunofluorescence staining images for identifying stem cell markers of neural stem cells induced by direct cross-differentiation from human adult fibroblasts.

FIG. 9 shows the qRT-PCR results for neural stem cell markers of neural stem cells directly cross-differentiated from human adult fibroblasts.

FIG. 10 shows the culturing results (colonies) of neural stem cells induced by direct cross-differentiation from human fibroblasts of patients with Huntington's disease.

FIG. 11 is a set of immunofluorescence staining images for identifying stem cell markers for neural stem cells induced by direct cross-differentiation from human fibroblasts of patients with Huntington's disease.

FIG. 12 shows the qRT-PCR results for neural stem cell markers of neural stem cells directly cross-differentiated from human fibroblasts of patients with Huntington's disease.

FIG. 13 is a diagram showing the comparison in neural stem cells conversion efficiency in human fibroblasts (neonatal fibroblasts, adult fibroblasts, human fibroblasts of patients with Huntington's disease).

FIG. 14 shows the results of GABAergic neuron differentiation to confirm the differentiation ability of neural stem cells induced in the present invention.

FIG. 15 shows the immunocytochemistry results of differentiated GABAergic neurons, confirming that the initial marker for neurons, Tuj1, and a GABA marker indicating GABAergic neurons are expressed.

FIG. 16 shows the results of inducing direct cross-differentiation into neural stem cells by injecting 375 ng of OCT4 mRNA and 125 ng of SOX2 mRNA.

FIG. 17 shows the expression of stem cell genes in neural stem cells induced by differentiation by transducing OCT4 and SOX2 mRNA.

FIG. 18 is a set of images showing the process of culturing neural stem cells induced by differentiation by transfecting miRNA302/367 and the results thereof.

FIG. 19 shows the expression of stem cell genes of neural stem cells induced by differentiation by transfecting miRNA302/367.

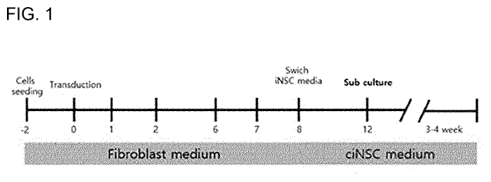

### MODES OF THE INVENTION

In one embodiment of the present invention, the present invention relates to a direct cross-differentiation-inducing composition for inducing direct conversion into neural stem cells from fibroblasts, which includes Sendai virus and one or more small molecule compounds selected from the group consisting of thiazovivin, valproic acid, purmorphamine, A8301, SB43154, CHIR99021, 5-aza-2′-deoxycytidine, and DZNep.

In another embodiment of the present invention, the present invention relates to a method of preparing neural stem cells, which includes culturing human fibroblasts in a medium including Sendai virus and one or more small molecule compounds selected from the group consisting of thiazovivin, valproic acid, purmorphamine, A8301, SB43154, CHIR99021, 5-aza-2′-deoxycytidine, and DZNep.

In still another embodiment of the present invention, the present invention relates to a cell therapeutic agent, which includes the direct cross-differentiation-inducing composition; or neural stem cells prepared by the method of preparing neural stem cells which includes culturing human fibroblasts in a medium including the composition. In yet another embodiment of the present invention, the present invention relates to a pharmaceutical composition for treating a brain disease selected from the group consisting of stroke, apoplexy, cerebral hemorrhage, cerebral infarction, Alzheimer's disease, dementia, Huntington's disease, Parkinson's disease, multiple sclerosis, multiple neurotrophy, epilepsy, Pick's disease, and Creutzfeldt-Jakob's disease, which includes the cell therapeutic agent.

Hereinafter, examples will be described in detail to specifically explain the present specification. However, the examples according to the present invention may be modified in a variety of different forms, and it should not be construed that the scope of the present invention is limited to the following examples. The examples of the present invention are provided to more completely explain the present invention to those of ordinary skill in the art.

### Example 1. Induction of Direct Cross-Differentiation into Neural Stem Cells from Human Neonatal Fibroblasts

To induce human fibroblasts into neural stem cells, 1×10⁵ human neonatal fibroblasts were prepared in a 60-mm dish, and the prepared cells were cultured by adding 8 types of reprogramming-related small molecule compounds (thiazovivin, valproic acid, purmorphamine, A8301, SB431542, CHIR99021, 5-aza-2′-deoxycytidine, and DZNep), and Sendai virus (OSKM, CytoTune-IPS 2.0 Sendai reprogramming kit) to a neurobasal medium composed of DMEM/F12, N2 supplement, B27 supplement, bFGF, and EGF. The properties of each of the 8 types of small molecule compounds have been described in a prior patent (PCT/KR2016/003819).

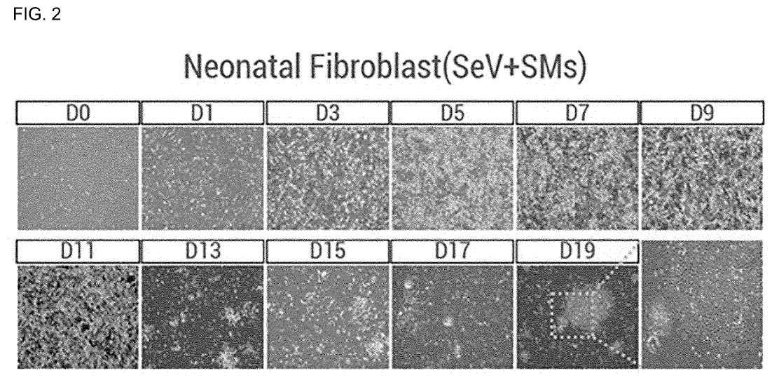

Differentiation was induced in the medium for 19 days, and the process of differentiating fibroblasts into neural stem cells while exchanging the medium every 2 or 3 days was then confirmed as shown in FIG. 2. As shown in FIG. 2, on day 13 of culture, small neural stem cell-like cells were observed, colonies of the neural stem cells were confirmed on day 15, and colonies that are sufficient to secure a single colony of the neural stem cells were detected on day 19.

In previous studies, when Sendai virus was not added, the period for direct cross-differentiation of fibroblasts into neural stem cells was approximately 60 days, but it was found that the period was significantly shortened when Sendai virus was also treated.

### Example 2. Comparison of Direct Cross-Differentiation Efficiency of Small Molecule Compound and Sendai Virus

To compare the direct cross-differentiation efficiency of the composition of the medium including Sendai virus and a small molecule compound of the present invention with previous study results using only a small molecule compound, a comparative experiment was conducted with the combinations shown in Table 1 below.

TABLE 1

| | | |
|---|---|---|
| 1 | Only SeV | Direct conversion into neural stem cells using only |
| | | SeV (OSKM) |
| 2 | 3SMs | Direct conversion into neural stem cells using only |
| | | SB431542, CHIR99021, and hLIF |
| 3 | 4SMs | Direct conversion into neural stem cells using only |
| | | A8301, purmorphamine, and valproic acid |
| 4 | 8SMs | Direct conversion into neural stem cells using only |
| | | thiazovivin, valproic acid, purmorphamine, A8301, |
| | | SB431542, CHIR99021, 5-aza-2′-deoxycitidine, |
| | | and DZNep |
| 5 | SeV + 3SMs | Direct conversion into neural stem cells using medium |
| | | under the conditions of 2), treated with SeV(OSKM) |
| 6 | SeV + 4SMs | Direct conversion into neural stem cells using medium |
| | | under the conditions of 3), treated with SeV(OSKM) |
| 7 | SeV + 8SMs | Direct conversion into neural stem cells using medium |
| | | under the conditions of 4), treated with SeV(OSKM) |

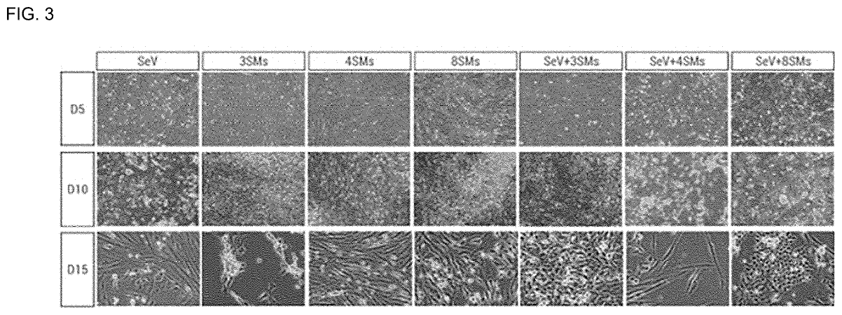

As a result of inducing direct cross-differentiation of human neonatal fibroblasts under the condition of a medium including small molecule compounds with 7 different conditions, no neural stem cell-like colonies were observed under the different conditions as shown in FIG. 3, but under the condition of the medium in 7), neural stem cell-like cells started to appear on day 13, and small neural stem cell colonies were detected on day 15.

### Example 3. Confirmation of Expression of Marker of Directly Cross-Differentiated Neural Stem Cells

To confirm whether human neural stem cells induced by Sendai virus and 8 types of small molecule compounds exhibit the properties of general neural stem cells, the expression of markers for neural stem cells was confirmed through qRT-PCR.

First, neural stem cells were induced by treating human neonatal fibroblasts with Sendai virus, and culturing them in a neurobasal medium composed of DMEM F12, N2 supplement, B27 supplement, bFGF, and EGF for 19 days (Only SeV iNSC). Human fibroblasts were treated with Sendai virus, 8 types of small molecule compounds such as thiazovivin, valproic acid, purmorphamine, A8301, SB431542, CHIR99021, 5-aza-2′-deoxycytidine, and DZNep were added to in a neurobasal medium composed of DMEM F12, N2 supplement, B27 supplement, bFGF, and EGF, and then cultured for 19 days to induce neural stem cells (SeV+8SMs iNSC).

In addition, neural stem cells were induced by culturing human fibroblasts in a medium in which 8 types of small molecule compounds such as thiazovivin, valproic acid, purmorphamine, A8301, SB431542, CHIR99021, 5-aza-2′-deoxycytidine, and DZNep were added to a neurobasal medium composed of DMEM F12, N2 supplement, B27 supplement, bFGF and EGF for 60 days without treatment with Sendai virus (Only 8SMs iNSC).

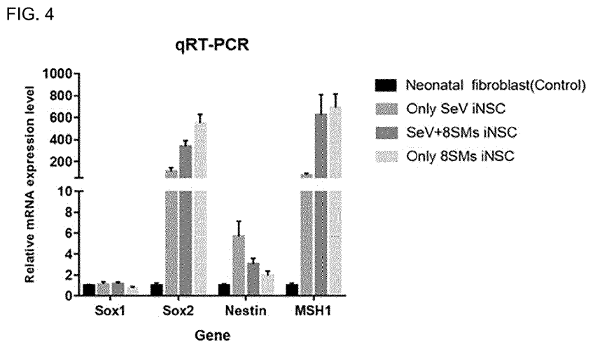

The expression levels of neural stem cell markers, such as Sox2, Nestin, and MSH1 in the neural stem cells (Only SeV iNSC/SeV+8SMs iNSC/Only 8SMs iNSC) induced by the above method were confirmed through qRT-PCR. As a result, as shown in FIG. 4, compared to the control, neonatal fibroblasts, it was confirmed that neural stem cell markers increased approximately 200 to 600-fold in the induced neural stem cells. In addition, in SeV+8SMs iNSC and Only 8SMs iNSC, compared to Only SeV iNSC, 3-fold higher expression levels of Sox2 and MSH1 were exhibited. However, in terms of the period of inducing differentiation, in the case of SeV+8SMs iNSC, a large quantity of neural stem cells could be secured after 19 days (Example 1), confirming that, as shown in the prior invention (PCT/KR2016/003819), neural stem cells are induced about 40 days faster, compared to when only 8 types of small molecule compounds were used.

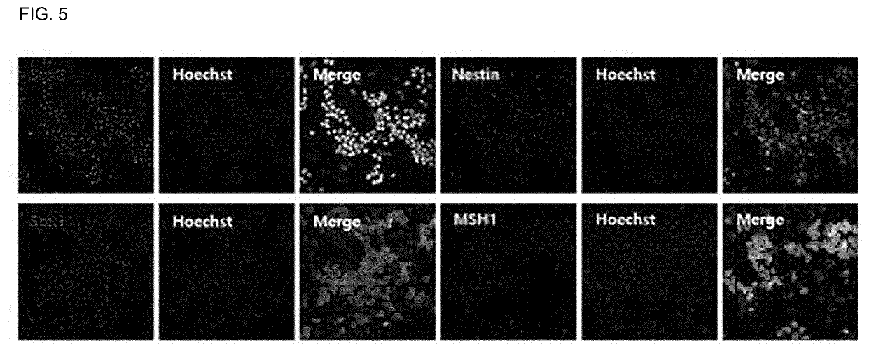

In addition, for the neural stem cell markers induced from the human neonatal fibroblasts, as a result of immunocytochemistry (ICC) performed on neural stem cell markers, such as Sox1, Sox2, MSH1, and Nestin, all neural stem cell markers were detected in the selected neural stem cells, and the differentiated cells were identified as neural stem cells (FIG. 5).

### Example 4. Confirmation of Chromosomal Stability Through Subculture

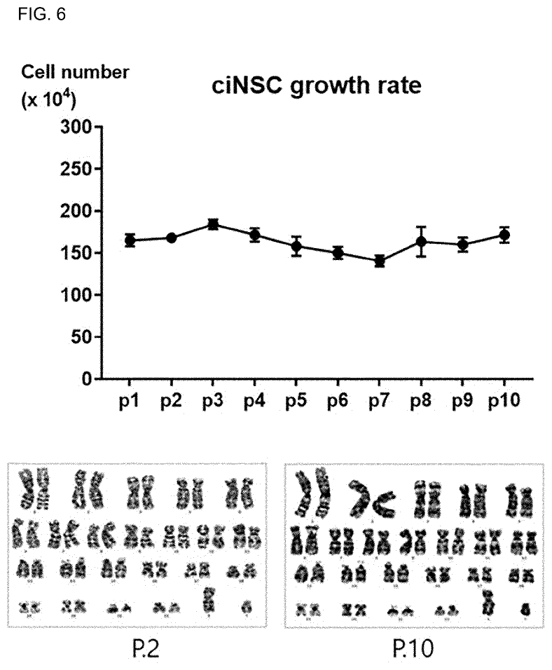

Subculture was conducted to increase the stem cell property of the neural stem cells induced from the human neonatal fibroblasts. To confirm genetic stability, a total of 10 passages of subculture were conducted, thereby confirming the chromosomal stability of the neural stem cells obtained at the 2nd passage and the 10th passage. As a result, as shown in FIG. 6, it was confirmed that all stem cells have normal chromosomal stability.

### Example 5. Induction of Neural Stem Cells Using Adult Fibroblasts

In Examples 1 to 4, neural stem cells were induced using neonatal fibroblasts, but to confirm whether neural stem cells are induced from adult fibroblasts in the same manner as above, neural stem cells were induced in the same manner as in Examples 1 to 4 using adult fibroblasts and their characteristics were confirmed.

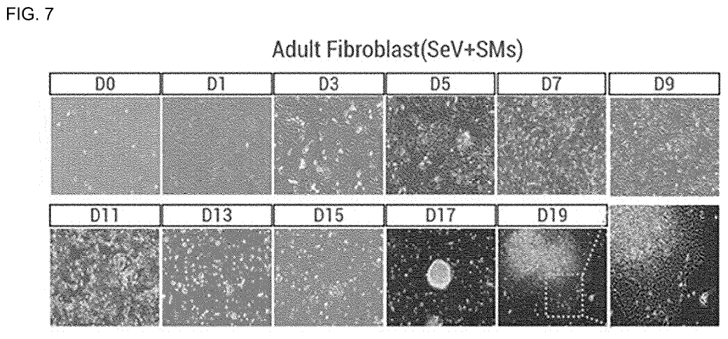

As a result, as shown in FIG. 7, as a result of inducing and observing differentiation for 19 days as described in Example 1, the morphology of neural stem cells began to be observed on day 13, and colonies of neural stem cells could be identified on day 19.

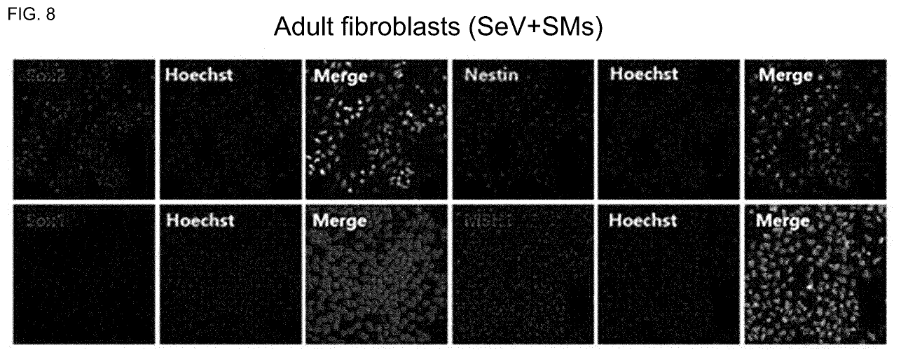

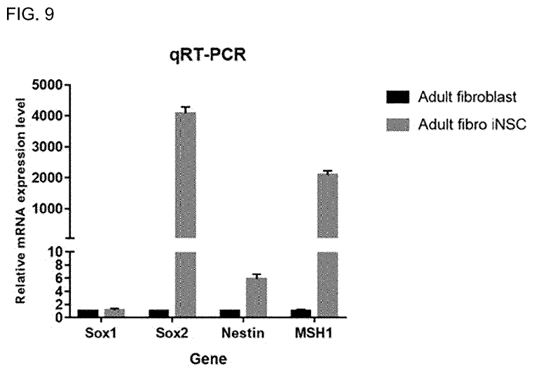

In addition, as a result of performing immunocytochemistry on the induced neural stem cells, neural stem cell markers such as Sox1, Sox2, MSH1, and Nestin could be confirmed (FIG. 8), and as a result of qRT-PCR, it was observed that the expression of Sox2, Nestin, and MSH1 was exhibited at higher levels compared to adult fibroblasts (FIG. 9), and confirmed that neural stem cells can be successfully induced using adult fibroblasts.

### Example 6. Induction of Neural Stem Cells Using Fibroblasts of Patients with Huntington's Disease

To confirm whether neural stem cells can be induced through direct cross-differentiation using fibroblasts of actual brain disease patients, neural stem cells were induced in the same manner as in Examples 1 to 4, and their properties were confirmed.

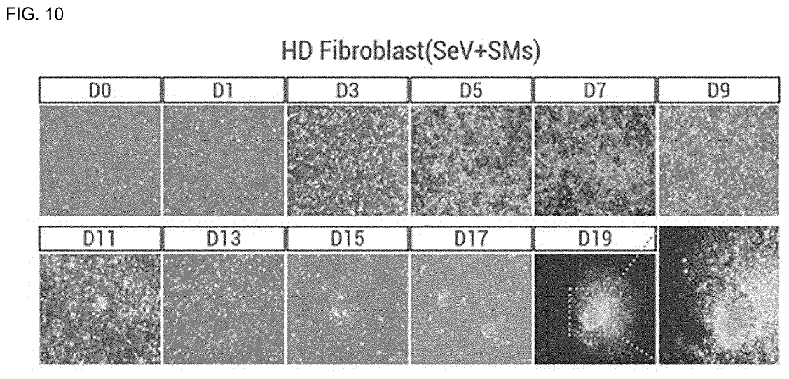

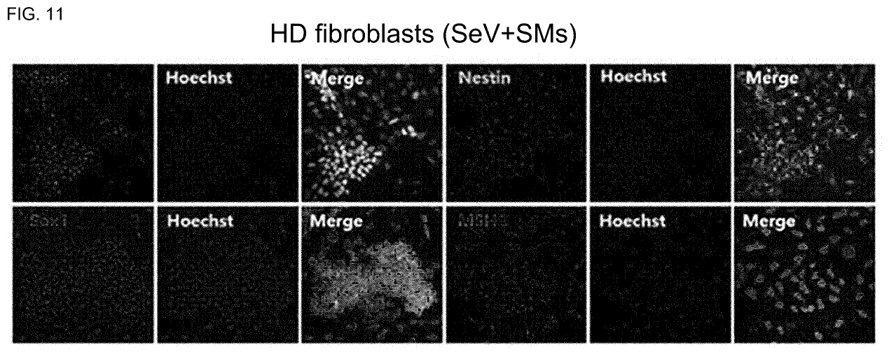

As shown in FIG. 10, as a result of inducing and observing differentiation for 19 days, the morphology of neural stem cells began to be observed on day 13, and colonies of neural stem cells were identified on day 19.

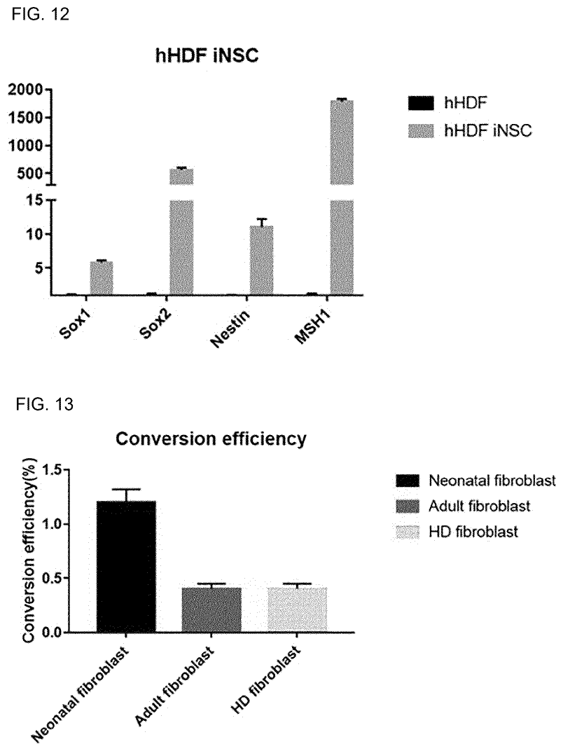

In addition, as a result of performing immunocytochemistry on the induced neural stem cells, neural stem cell markers such as Sox1, Sox2, MSH1, and Nestin could be confirmed (FIG. 11), and qRT-PCR revealed that the expression of Sox2, Nestin, and MSH1 was exhibited at higher levels compared to adult fibroblasts (FIG. 12), confirming that neural stem cells can be successfully induced using adult fibroblasts.

As result, for all of human neonatal fibroblasts, adult fibroblasts, and fibroblasts of Huntington's disease patients, it was confirmed that neural stem cells were successfully induced using a medium including Sendai virus and 8 types of small molecule compounds, and as a result of comparing the conversion efficiency of each type of fibroblasts, as shown in FIG. 13, the conversion efficiency of the human neonatal fibroblasts was approximately 1.2%, and the conversion efficiencies of the adult fibroblasts and the fibroblasts of Huntington's disease patients were approximately 0.4%.

### Example 7. Confirmation of Differentiation Ability of Induced Neural Stem Cells

To confirm the differentiation ability of neural stem cells induced in the above example, differentiation into GABAergic neurons was induced in vitro. GABAergic neurons secrete γ-aminobutyric acid (GABA) in the brain, and GABA is a non-protein amino acid distributed in nature and is known as a neurotransmitter present in the brain or spinal cord of mammals.

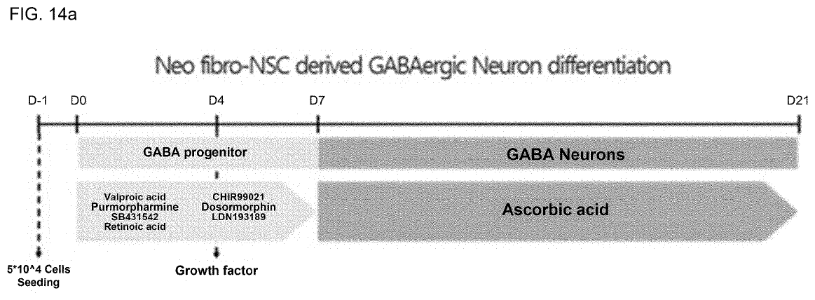

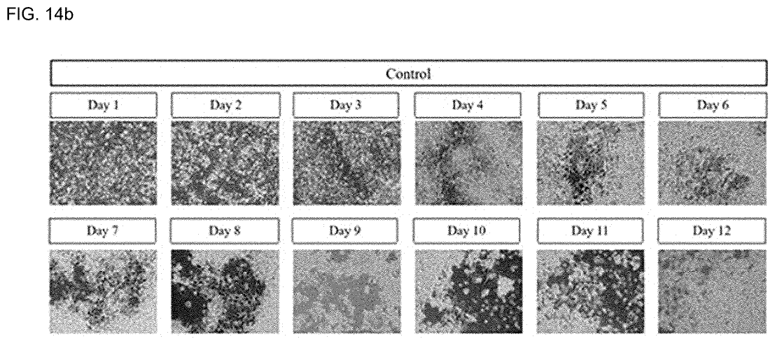

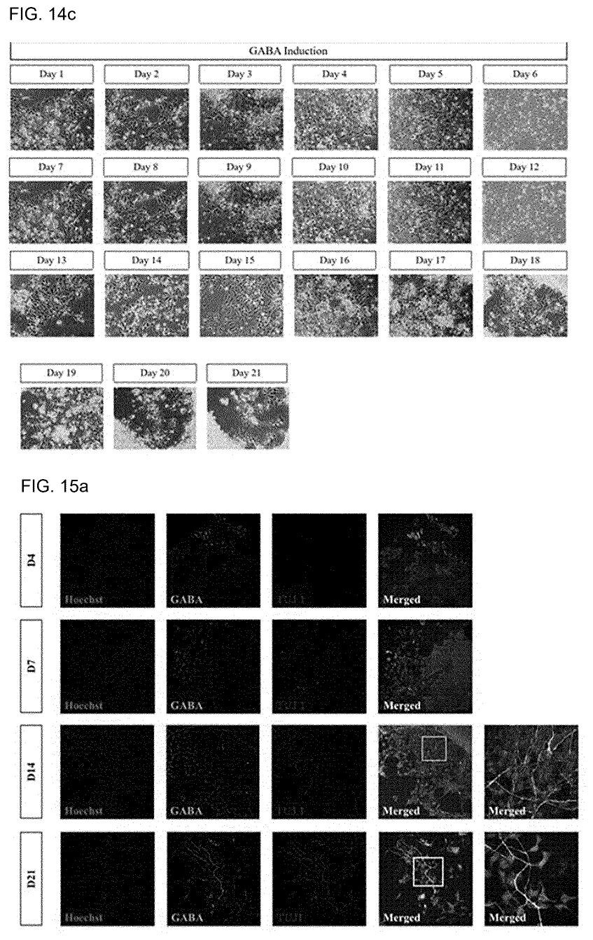

The neural stem cells induced in the above example were seeded at 5×10⁴ cells/well on day 1 in a 24-well plate coated with poly-L-ornithine/fibronectin (PLO/FN), and cultured in a medium in which DMEM F12 and neurobasal media were mixed at 1:1 and which was supplemented with 0.5×N2 supplement, 0.5×B27 supplement, 1×MEM-NEAA, 100 μg/mL penicillin and streptomycin, 10 μM valpronic acid, 0.65 μM purmorphamine, 2 μM SB431542, 0.1 μM retinoic acid 0.1 μM, 1 μM CHIR99021, 200 nM dorsormorphin, 100 nM LDN193189, and 200 μM ascorbic acid for 0 to 7 days. On day 7 to day 21, the cells were cultured in a medium in which DMEM F12 and neurobasal media were mixed at 1:1 and which was supplemented with 0.5×N2 supplement, 0.5×B27 supplement, 1×MEM-NEAA, 100 μg/mL penicillin and streptomycin, and 200 μM ascorbic acid, and it was confirmed that the cells differentiated into neurons (GABA neurons) with a neuron-like shape by day 21 of differentiation (FIG. 14).

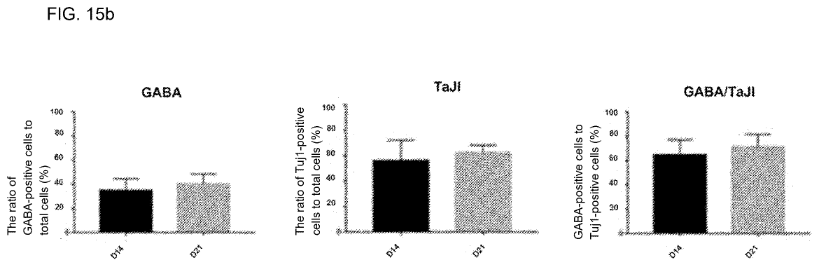

In addition, as a result of performing immunocytochemistry on the differentiated GABAergic neurons, it was confirmed that the early marker of neurons, Tuji, and a GABA marker indicating GABAergic neurons were expressed (FIG. 15). That is, it was confirmed that neural stem cells induced by direct cross-differentiation using the medium including Sendai virus and 8 types of small molecule compounds of the present invention can successfully differentiate into neurons.

### Example 8. Induction of Neural Stem Cells Through Gene Introduction Using mRNA

In the induction of direct cross-differentiation into neural stem cells using human neonatal fibroblasts, to confirm the influence of a transcription factor OCT4 for maintaining the pluripotency of stem cells and the factor SOX2 required for converting somatic cells into induced pluripotent cells, after directly transfecting human neonatal fibroblasts with stem cell genes, such as OCT4 mRNA, and SOX2 mRNA to introduce the genes, direct cross-differentiation into neural stem cells was induced using the iNSC medium of the present invention.

The human neonatal fibroblasts were seeded at 5×10⁴ cells/well in a 24-well plate, and incubated overnight in a DMEM+10% FBS medium. The next day, Lipofectamine messengerMAX reagent and an OptiMEM medium (25 μL), suitable for each condition, were vortexed and incubated at room temperature for 10 minutes (Mixture 1). OCT4 mRNA and SOX2 mRNA were diluted to a required amount in an Opti-MEM medium (50 μL) (Mixture 2), and Mixture 1 and Mixture 2 were mixed together and incubated at room temperature for 5 minutes. Afterward, the resulting mixture was added to each 24-well plate to perform transfection for 6 hours or 24 hours. The medium was then exchanged with an iNSC medium to induce direct cross-differentiation.

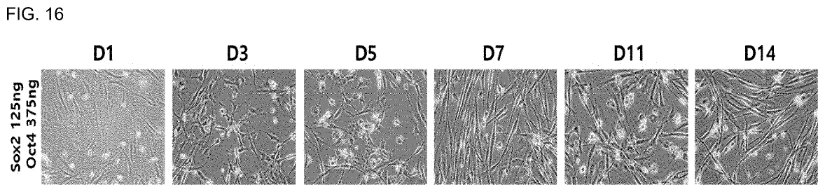

As a result of adding DMEM F12, N2 supplement, B27 supplement, thiazovivin, valproic acid, purmorphamine, A8301, SB431542, and CHIR99021 to the iNSC and further culturing the cells for 14 days, as shown in FIG. 16, it was shown that the cells decreased in size until day 3 of transfection and then became elongated, but were not changed into neural stem cells even after day 14.

The human neonatal fibroblasts induced to differentiate under the SOX2+OCT+8SMs conditions were subjected to qRT-PCR to confirm highly-expressed RNA on day 7 and day 14, and the expression levels thereof were compared.

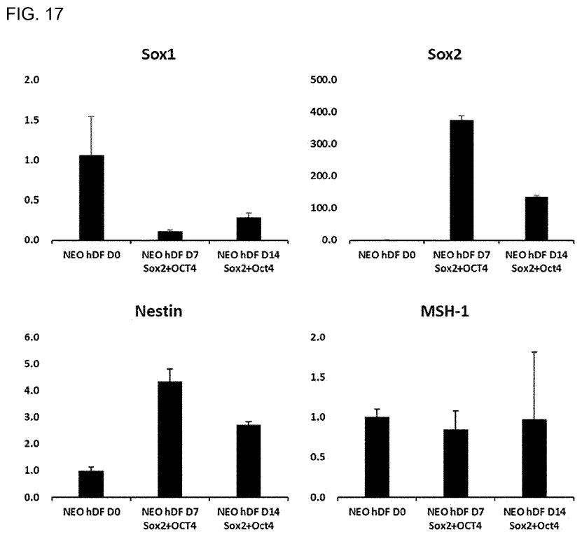

Human neonatal fibroblasts (NEOhDF DO) were used as a control, and it was confirmed that the SOX2 expression level increased 100-fold or more under the conditions of NEOhDF D7 SOX2+OCT4 and NEOhDF D14 SOX2+OCT4 (FIG. 17).

### Example 9. Induction of Neural Stem Cells Using miRNA

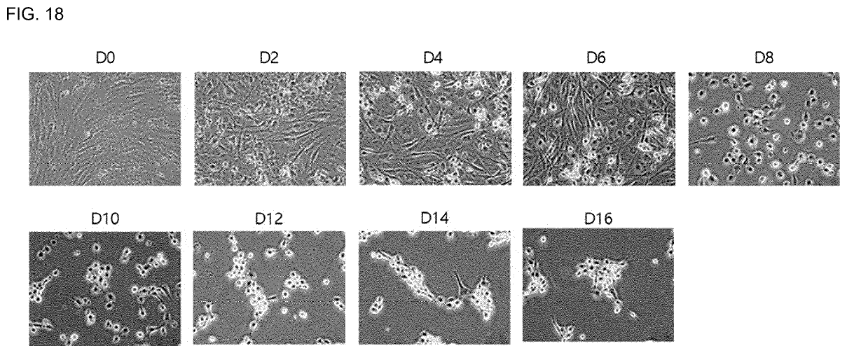

The experiment of inducing direct cross-differentiation of neural stem cells was performed by inhibiting the NR2F2 gene, which suppresses the OCT4 gene through miRNA302/367 expression. Human Huntington dermal fibroblasts were transfected with an miRNA302/307 cluster to regulate the gene, and direct cross-differentiation into neural stem cells was induced using an existing iNSC medium, thereby confirming induction efficiency. The human Huntington dermal fibroblasts were seeded at 2×10⁵ cells/well in a 6-well plate, 5 μL of Lipofectamine RNAiMAX and 5 nM miRNA302/307 were added, and incubated at room temperature for 1 minutes. The cells were then mixed with an Opti-MEM medium (1 mL) and transfected for 5 hours. The medium was exchanged with DMEM+media to incubate the cells for 1 day (T1), two days (T2) and three days (T3), and then exchanged again with a medium including 8 SMs to incubate the cells for 16 days, followed by observing differentiation. As a result of observing the cells on day 7 of subculture, the cell shape changed to a smaller and rounder shape, but the cell volume was reduced and the cells did not proliferate (FIG. 18).

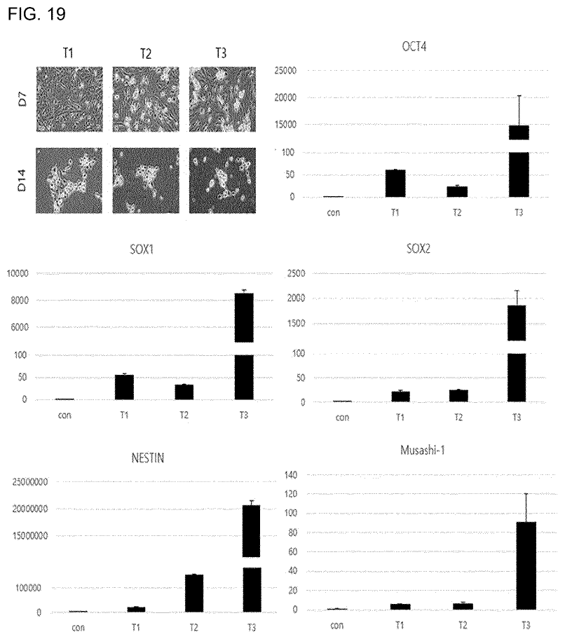

In addition, to confirm the neural stem cell gene expression in the cultured cells, on day 7 and day 14 of expression, an RT-PCR experiment was performed using OCT4, SOX1, SOX2, NESTIN, and Musashi-1. As a result, it was confirmed that the expression of stem cell-related markers was most highly exhibited on day 3 compared to the control. The increased expression of the related markers confirmed that the miRNA302/367 cluster helps direct cross-differentiation into stem cells (FIG. 19).

In the above, the present invention was described with reference to embodiments. It will be understood by those of ordinary skill in the art that the present invention can be implemented in modified forms without departing from the essential features of the present invention. Therefore, the disclosed embodiments should be considered from a descriptive perspective, rather than a restrictive perspective. The scope of the present invention is shown in the claims rather than the foregoing description, and all differences within the equivalent range thereto will be construed as being included in the present invention.

1. A direct cross-differentiation-inducing composition for inducing direct conversion into neural stem cells from fibroblasts, comprising:
one or more small molecule compounds selected from the group consisting of thiazovivin, valproic acid, purmorphamine, A8301, SB43154, CHIR99021, 5-aza-2′-deoxycytidine, and DZNep, and Sendai virus.

2. The composition of claim 1, further comprising:
a medium for cell culture.

3. The composition of claim 1, wherein the Sendai virus includes a Yamanaka factor.

4. The composition of claim 1, wherein the fibroblasts are derived from a human.

5. The composition of claim 1, wherein the neural stem cells differentiate into one or more selected from the group consisting of an astrocyte, an oligodendrocyte, a neuron, a dopamine neuron, a GABA neuron, a motor neuron, and a choline neuron.

6. A method of preparing neural stem cells, comprising:
culturing human fibroblasts in a medium including one or more small molecule compounds selected from the group consisting of thiazovivin, valproic acid, purmorphamine, A8301, SB43154, CHIR99021, 5-aza-2′-deoxycytidine, and DZNep, and Sendai virus.

7. The method of claim 6, wherein the Sendai virus includes a Yamanaka factor.

8. The method of claim 6, wherein the medium is DMEM/F12 including N2, B27, bFGF, and EGF.

9. The method of claim 6, wherein the culturing is performed for 10 to 20 days.

10. The method of claim 6, wherein the neural stem cells differentiate into one or more selected from the group consisting of an astrocyte, an oligodendrocyte, a neuron, a dopamine neuron, a GABA neuron, a motor neuron, and a choline neuron.

11. A cell therapeutic agent, comprising:
the composition of claim 1; or
neural stem cells prepared by a method of culturing human fibroblasts in a medium including the one or more small molecule compounds selected from the group consisting of thiazovivin, valproic acid, purmorphamine, A8301, SB43154, CHIR99021, 5-aza-2′-deoxycytidine, and DZNep, and Sendai virus.

12. A pharmaceutical composition for treating a brain disease, comprising the cell therapeutic agent of claim 11.

13. The pharmaceutical composition of claim 12, wherein the brain disease is selected from the group consisting of stroke, apoplexy, cerebral hemorrhage, cerebral infarction, Alzheimer's disease, dementia, Huntington's disease, Parkinson's disease, multiple sclerosis, multiple neurotrophy, epilepsy, Pick's disease, and Creutzfeldt-Jakob's disease.

14. A method for treating a brain disease, comprising administering to a subject in need thereof the cell therapeutic agent of claim 11.
